(19) United States
(12) Patent Application Publication
(10) Pub. No.: US 2020/0103400 A1
(43) Pub. Date: Apr. 2, 2020

(54) INTEGRATED OPTO NANO ELECTRONIC (iONE) SENSING

(71) Applicant: ACCURE HEALTH, INC., Cambridge, MA (US)

(72) Inventors: Liyun Sang, Cambridge, MA (US); Qiang Liu, Cambridge, MA (US)

(21) Appl. No.: 16/701,127

(22) Filed: Dec. 2, 2019

Related U.S. Application Data
(63) Continuation of application No. PCT/US2018/037171, filed on Jun. 12, 2018.
(60) Provisional application No. 62/525,857, filed on Jun. 28, 2017.

(51) Int. Cl. G01N 33/543; G01N 27/327

(52) U.S. Cl. CPC G01N 33/54346; G01N 27/3278; G01N 27/3271; G01N 33/54326

(57) ABSTRACT

The present disclosure provides a method for signal amplification of electrochemical sensing of target analytes. The method includes irradiating a complex comprising a target analyte and a nanoparticle, with a light source in proximity to an electrochemical sensor. The method further includes conducting electrochemical sensing, by the electrochemical sensor, with respect to the irradiated complex to determine a presence or quantity of the target analyte. The present disclosure also provides a system for detecting a target analyte. The system includes an electrochemical sensor, a potentiostat, and a light source. The electrochemical sensor includes a plurality of electrodes and configured to perform an assay to detect a presence or quantity of a target analyte in a sample. The potentiostat is operatively coupled to the electrochemical sensor. The light source is configured to irritate a complex including the target analyte formed during the assay.

### CROSS-REFERENCE TO RELATED APPLICATIONS

This application is continuation of International Application No. PCT/US2018/037171, filed on Jun. 12, 2018, entitled “INTEGRATED OPTO NANO ELECTRONIC (iONE) SENSING,” which claims the benefit of priority to U.S. Provisional Application No. 62/525,857, filed on Jun. 28, 2017, both of which are hereby incorporated by reference in their entireties.

### BACKGROUND

The present disclosure provides methods and systems for electrochemical sensing of a target analyte.

Biological samples are often examined for the presence and prevalence of particular analytes, such as cells, vesicles, proteins, peptides, lipids, nucleic acids, metabolites, and other small molecules. The presence and prevalence of specific analytes can provide insight into a particular biological or pathogenic process, a progression of a particular disease, or some other biological condition of a subject. The presence and prevalence of specific analytes can provide insight into the composition of a particular substance or material.

A need exists for an amplification method for electrochemical sensing of different target analytes with enhanced sensitivity.

### SUMMARY

Embodiments of the disclosure provide a method for ultra sensitive electrochemical sensing of target analytes by harnessing light. A complex comprising a nanoparticle and a target analyte is irradiated with a light source in proximity to an electrochemical sensor. Electrochemical sensing is conducted, by the electrochemical sensor, with respect to the irradiated complex to determine a presence or quantity of the target analyte.

Embodiments of the disclosure also provide a method for detecting a target analyte in a sample. A first binding moiety is provided on a substrate to a sample comprising a target analyte. The first binding moiety specifically binds to the target analyte. The target analyte in the sample is allowed to bind with the first binding moiety on the substrate, thereby forming a first complex comprising the target analyte and the first binding moiety on the substrate. A nanoparticle comprising a second binding moiety that binds to the target analyte is provided. The nanoparticle is conjugated to a reporter enzyme. The first complex is allowed to bind with the second binding moiety of the nanoparticle, thereby forming a second complex comprising the first complex and the second binding moiety of the nanoparticle. An electron mediator is introduced into the electrochemical sensor. The second complex is applied to a sample detection region of a first electrode. The first electrode is electrically coupled to a potentiostat. The second complex in the sample detection region is irradiated with a light source. An oxidation-reduction reaction is introduced between the electron mediator and the reporter enzyme. An output of the potentiostat is monitored to determine a presence or quantity of the target analyte in the sample.

Embodiments of the disclosure further provide a method for detecting a target analyte in a fluid sample. A plurality of magnetic beads are provided to a first fluid sample. The plurality of magnetic beads comprise first binding moieties that specifically bind to the target analyte. The first binding moieties of the plurality of magnetic beads are allowed to bind to the target analyte in the first fluid sample. The plurality of magnetic beads are transferred from the first fluid sample to a second fluid sample. The second fluid sample comprises a plurality of nanoparticles. The plurality of nanoparticles comprise second binding moieties that bind to the target analyte. The plurality of nanoparticles are conjugated to a plurality of reporter enzymes. The second binding moieties of the plurality of nanoparticles are allowed to bind to the target analyte bound to the first binding moieties of the plurality of magnetic beads. The second fluid sample comprising the plurality of magnetic beads and the plurality of nanoparticles with a solution comprising a plurality of electron mediators are combined to obtain a third fluid sample. The third fluid sample is provided to a sample detection region of a first electrode. The first electrode is electrically coupled to a potentiostat. The third fluid sample is irradiated with a light source. An oxidation-reduction reaction is introduced between the plurality of electron mediators and the reporter enzymes in the third fluid sample. An output of the potentiostat is monitored to determine a presence or quantity of the target analyte in the third fluid sample.

Embodiments of the disclosure also provide a method for detecting a target analyte in a sample. A first binding moiety is provided on a substrate to a sample comprising a target analyte. The first binding moiety specifically binds to the target analyte. The target analyte in the sample is allowed to bind with the first binding moiety on the substrate, thereby forming a first complex comprising the target analyte and the first binding moiety on the substrate. A nanoparticle comprising a second binding moiety that binds to the target analyte is provided. The first complex is allowed to bind with the second binding moiety of the nanoparticle, thereby forming a second complex comprising the first complex and the second binding moiety of the nanoparticle. Hydrogen ions are introduced into the electrochemical sensor. The second complex is applied to a sample detection region of a first electrode. The first electrode is electrically coupled to a potentiostat. The second complex in the sample detection region is irradiated with a light source. An oxidation-reduction reaction involving the hydrogen ions and the nanoparticle is introduced. An output of the potentiostat is monitored to determine a presence or quantity of the target analyte in the sample.

Embodiments of the disclosure further provide a method for detecting a target analyte in a fluid sample. A plurality of magnetic beads are provided to a first fluid sample. The plurality of magnetic beads comprise first binding moieties that specifically bind to the target analyte. The first binding moieties of the plurality of magnetic beads are allowed to bind to the target analyte in the first fluid sample. The plurality of magnetic beads are transferred from the first fluid sample to a second fluid sample. The second fluid sample comprises a plurality of nanoparticles. The plurality of nanoparticles comprise second binding moieties that bind to the target analyte. The second binding moieties of the plurality of nanoparticles are allowed to bind to the target analyte bound to the first binding moieties of the plurality of magnetic beads. The second fluid sample comprising the plurality of magnetic beads and the plurality of nanoparticles with an acid medium are combined to obtain a third fluid sample. The third fluid sample is provided to a sample detection region of a first electrode. The first electrode is electrically coupled to a potentiostat. The third fluid sample is irradiated with a light source. An oxidation-reduction reaction involving hydrogen ions and the nanoparticles is introduced in the third fluid sample. An output of the potentiostat is monitored to determine a presence or quantity of the target analyte in the third fluid sample.

Embodiments of the disclosure further provide a system for detecting a target analyte. The system includes an electrochemical sensor comprising a plurality of electrodes, the electrochemical sensor configured to perform an assay to detect a presence or quantity of a target analyte in a sample. The system also includes a potentiostat operatively coupled to the electrochemical sensor. The system further includes a light source configured to irritate a complex comprising the target analyte formed during the assay.

### BRIEF DESCRIPTION OF THE DRAWINGS

The accompanying drawings, which are incorporated herein and form a part of the specification, illustrate the present disclosure and, together with the description, further serve to explain the principles of the disclosure and to enable a person skilled in the pertinent art to make and use the disclosure.

FIG. 1 is a block diagram illustrating an example of an electrochemical sensing system for detecting a target analyte.

FIG. 2A is a schematic diagram illustrating an example of the integrated Opto Nano Electronic (iONE) sensing method.

FIG. 2B is a schematic diagram illustrating another example of the integrated Opto Nano Electronic (iONE) sensing method.

FIG. 3A is a flow chart diagram illustrating one example of the iONE sensing method.

FIG. 3B is a flow chart diagram illustrating another example of the iONE sensing method.

FIG. 4A is a flow chart diagram illustrating still another example of the iONE sensing method.

FIG. 4B is a flow chart diagram illustrating yet another example of the iONE sensing method.

FIGS. 5A-5C depict effects of light irradiation and nanoparticles on an oxidation-reduction reaction.

FIGS. 6A-6C depict effects of light irradiation of different wavelengths on an oxidation-reduction reaction.

FIG. 7 depicts effect of light irradiation and nanoparticles in the detection of HVEM expression in urine.

FIGS. 8A-8B depict effects of light irradiation and nanoparticles in the detection of CD47 expression in tumor-derived extracellular vesicle (EV).

### DETAILED DESCRIPTION

Unless defined otherwise, all technical and scientific terms used herein have the same meaning as commonly understood by one of ordinary skill in the art to which the disclosure pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

As used in this specification and the appended claims, the singular forms “a,” “an,” and “the” include plural referents unless the context clearly dictates otherwise. Thus, for example, reference to “an analyte” includes a plurality of such analytes, and the like.

The term “about” as used herein indicates the value of a given quantity varies by ±10% of the value.

Unless clearly indicated otherwise, ranges listed herein are inclusive.

A variety of additional terms are defined or otherwise characterized herein.

Methods for detecting a target analyte using iONE sensing are described herein. As used herein, the term “detect” includes identifying the presence or absence of one or more target analytes, and/or quantifying the amount and/or concentration of one or more target analytes in a sample.

One or more of the implementations described herein can be used to identify analytes such as cells, extracellular vesicles (EVs) such as microvesicles, membrane particles, apoptotic blebs or vesicles, or exosomes, proteins (e.g., secreted, transmembrane and cytosolic proteins, degraded proteins, post-translationally modified proteins), nucleic acids (e.g. mRNA, DNA, and microRNA among others), peptides, lipids, metabolites, and other molecules, either free-floating (e.g., in a serum or a solution) or expressed on the surface of a biological structure (e.g., on the surface of an extracellular vesicle or a cell).

In an example of an implementation, a sample is collected from a subject (e.g., a sample of biological fluid, such as blood, urine, saliva, CSF, ascites, and pleural fluid). The sample is processed using magnetic separation, such that particular analytes of interest (e.g., analytes indicative of a particular biological or pathogenic process, a particular progression of a particular disease, or some other biological condition) are isolated and/or concentrated near a sample probe of a measurement instrument. The presence and/or prevalence of these analytes in the sample are subsequently investigated using the measurement instrument via electrochemical detection. The resulting information can be used to provide more effective care to the subject (e.g., by enabling caretakers to make diagnoses and/or administer treatment in a more informed manner).

In another example of an implementation, a sample is collected from a substance. The sample is processed using magnetic separation, such that particular analytes of interest (e.g., analytes indicative of a particular material or antigen) are isolated and/or concentrated near a sample probe of a measurement instrument. The presence and/or prevalence of these analytes in the sample are subsequently investigated using the measurement instrument via electrochemical detection. The resulting information can be used to provide insight into the composition of the substance. As an example, samples of a food product can be analyzed for the presence or quantity of particular allergens, such that a consumer can make more informed choices regarding his diet.

In some embodiments, samples can be analyzed without the need to conduct extensive processing techniques (e.g., filtration or centrifugation), which may require specialized equipment. Thus, users can analyze samples more easily and/or in a more cost-effective manner. In some embodiments, users can examine each sample quickly, such that many samples can be efficiently examined for the presence or quantity of one analyte or multiple different analytes. In some embodiments, lay users can conduct an examination themselves without the assistance of an experienced technician, and without the need for expensive equipment.

System for Detecting a Target Analyte

According to one aspect, the present disclosure provides a system for detecting a target analyte, comprising: an electrochemical sensor comprising a plurality of electrodes, the electrochemical sensor configured to perform an assay to detect a presence or quantity of a target analyte in a sample; a potentiostat operatively coupled to the electrochemical sensor; and a light source configured to irritate a complex comprising the target analyte formed during the assay.

In some embodiments, the light source is a red light, a green light, a white light, a blue light, a yellow light or any combinations thereof.

In some embodiments, the light source is a laser diode or a light emitting diode. In some embodiments, the laser diode or light emitting diode emits a light having a wavelength between about 630 nm and about 680 nm. In some embodiments, the laser diode or light emitting diode emits a light having a wavelength around 520-535 nm. In some embodiments, the laser diode or light emitting diode emits a light having a wavelength around 585-595 nm. In some embodiments, the laser diode or light emitting diode emits a light having a wavelength around 620˜630 nm. In some embodiments, the power of the laser diode or light emitting diode is about 5 mW.

In some embodiments, the complex further comprises a nanoparticle conjugated to a reporter enzyme. In some embodiments, the nanoparticle is a gold nanoparticle (AuNP).

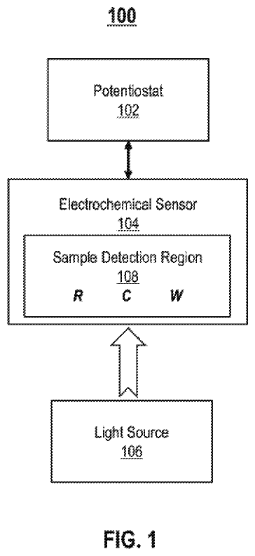

An example of a system 100 for detecting a target analyte is schematically shown in FIG. 1. System 100 includes a potentiostat 102, an electrochemical sensor 104, and a light source 106. System 100 may also include any other suitable components for sample handling, electrochemical sensing and/or light irradiation such as, but not limited to, fluidic components (e.g., pumps, valves, channels, etc.), electrical components (e.g., analog-to-digital converters, microcontroller units, digital-to-analog converters, storage, power sources, Bluetooth, Wi-Fi, etc.) and optical components (e.g., optical assembly). In some embodiments, system 100 may be an integrated handheld device for point-of-care test and diagnostics.

Electrochemical sensor 104 includes a sample detection region 108 in which electrochemical sensing can occur. Sample detection region 108 includes three electrodes: a reference electrode R, a counter electrode C, and a working electrode W. During operation of system 100, each of these electrodes is placed into contact with a fluid sample in sample detection region 108 to be analyzed. In some embodiments, a magnet assembly (e.g., placed adjacent to sample detection region 108, not shown) attracts magnetically labeled particles in the fluid sample to the electrodes (e.g., by inducing a magnetic field that extends through the electrodes).

Potentiostat 102 is operatively coupled to each of the electrodes R, C, W, and is configured, during operation, to maintain a pre-defined potential difference between the working electrode W and the reference electrode R. Further, potentiostat 102 is configured to measure a current induced from the working electrode W across the counter electrode C.

In some embodiments, potentiostat 102 may include two operational amplifiers (not shown). The first operational amplifier is electrically coupled to the reference electrode R and the counter electrode C and is configured to maintain the pre-defined potential difference between the working electrode W and the reference electrode R (e.g., by applying a potential to the working electrode W as a bias relative to the potential of the reference electrode R). In some embodiments, the potential can be between about −1.65 V and about 1.65 V. In some embodiments, the potential can be about −0.1 V. In some embodiments, the potential can be about −1 V. The second operational amplifier is electrically coupled to the working electrode W and is configured as a trans-impedance amplifier to convert a current induced from the working electrode W across counter electrode C to a voltage signal.

The electrodes can be made of various materials. In some embodiments, the working electrode W and the counter electrode C can be made, either partially or entirely, of a first material (e.g., gold or carbon), and the reference electrode R can be made, either partially or entirely, of a second material (e.g., silver and/or silver chloride). In some embodiments, different materials can be used for some or all of the electrodes. In some embodiments, different materials may increase the signal level by increasing the effective surface area of electrodes.

Although a three-electrode implementation is described with respect to FIG. 1, it is understood that other configurations can be used to measure an induced current within a sample. For example, configurations such as dual-working electrode configurations (e.g., an interdigitated electrode array (IDA)) or two-electrode configurations (e.g., excluding a counter electrode) can be used.

As shown in FIG. 1, light source 106 irradiates light to sample detection region 108. Light source 106 can be any natural or artificial source of illumination. For example, light source 106 includes, but is not limited to, light emitting diodes (LEDs), lasers (e.g., laser diodes), incandescence bulbs, fluorescent lamps, electrical gas-discharge lamps, mercury vapor lamps, etc. Light source 106 may be a standalone component that can be moved away or to electrochemical sensor 104 or an integrated component attached to electrochemical sensor 104. In some embodiments, light source 106 may emit light in various wavelengths at the same time or different times. For example, light source 106 may simultaneously emit light in more than one wavelength or sequentially emit light in more than one wavelength. In some embodiments, the duration of irradiating by light source 106 can also be controlled as desired, for example, from about 5 seconds to about 5 minutes. In some embodiments, the power of irradiating by light source 106 may be controlled as well.

Light emits from light source 106 may pass an optical assembly (not shown) before it reaches to sample detection region 108. The optical assembly may include, for examples, mirrors, lenses, waveguides, fibers, splitters, etc. The optical assembly may adjust the optical path of light traveling from light source 106 to sample detection region 108 and/or adjust (enhance or attenuate) the intensity of the light reaching to sample detection region 108.

Amplification Method for Electrochemical Sensing

According to one aspect, the present disclosure provides an amplification method for electrochemical sensing of a target analyte, the method comprising: irradiating a complex comprising a target analyte and a nanoparticle, with a light source in proximity to an electrochemical sensor; and conducting electrochemical sensing, by the electrochemical sensor, with respect to the irradiated complex to determine a presence or quantity of the target analyte.

Examples of target analytes include, but are not limited to, cells, EVs such as microvesicles, membrane particles, apoptotic blebs or vesicles or exosomes, nucleic acids (e.g., mRNA, DNA, and microRNA), proteins, peptides, lipids, metabolites, and other molecules, either free-floating (e.g., in a serum or a solution) or expressed on the surface of a biological structure (e.g., on the surface of an extracellular vesicle or a cell).

In some embodiments, the target analyte comprises extracellular vesicles. Examples of the extracellular vesicles include, but are not limited to exosomes, microvesicles, oncosomes, other vesicles, and apoptotic bodies.

In some embodiments, the target analyte comprises at least one of EPCAM, EGFR, HER2, MUC1, MUC2, MUC6, MUC5AC, GPC1, WNT2, CEP, CD24, GPA33, CA125, CD44, CD44v6, CEA, Mesothelin, Trop2, Grp94, SSTR2, CD166, CD133, MET, B7H3, CD63, CD9, CD81, CD2, CD3, CD14, CD45, CD47, CD52, CD68, CD73 HLA-ABC, CXCL10, CXCL9, HVEM, FGFR3, NUMA, HSP70, IL-3, TSP2, PD-L1, EGFRv3, EGFR T790M, IDH1 mutant, APC mutant, KRAS mutant, BRAF mutant, PIK3CA mutant, BRAC1/2 mutant, SMAD4 mutant, CDKN2 mutant, and PTEN mutant biomarkers.

In some embodiments, the target analyte comprises a cell, a protein, a peptide, a lipid, nucleic acids, small molecules, microbes, food antigens, a toxin, opioids, or a metabolite.

In some embodiments, the nanoparticle comprises at least one metal. In some embodiments, the metal is gold, silver, platinum, iron, or copper. In some embodiments, the nanoparticle is a gold nanoparticle. It is to be appreciated that the material of the nanoparticle is not limited to the examples above but can be any suitable materials or any combinations of suitable materials.

In some embodiments, the nanoparticle ranges in size from about 1 nm to about 1000 nm, about 1 nm to about 500 nm, about 1 nm to about 250 nm, about 1 nm to about 200 nm, about 1 nm to about 100 nm, about 1 nm to about 50 nm, about 1 nm to about 25 nm, about 1 nm to about 5 nm, about 5 nm to about 1000 nm, about 5 nm to about 500 nm, about 5 nm to about 250 nm, about 5 nm to about 200 nm, about 5 nm to about 100 nm, about 5 nm to about 50 nm, about 5 nm to about 25 nm, about 25 nm to about 1000 nm, about 25 nm to about 500 nm, about 25 nm to about 250 nm, about 25 nm to about 200 nm, about 25 nm to about 100 nm, about 25 nm to about 50 nm, about 50 nm to about 1000 nm, about 50 nm to about 500 nm, about 50 nm to about 250 nm, about 50 nm to about 200 nm, about 50 nm to about 100 nm, about 100 nm to about 1000 nm, about 100 nm to about 500 nm, about 100 nm to about 250 nm, about 100 nm to about 200 nm, about 200 nm to about 1000 nm, about 200 nm to about 500 nm, about 200 nm to about 250 nm, about 250 nm to about 1000 nm, about 250 nm to about 500 nm, or about 500 nm to about 1000 nm. In some embodiments, the nanoparticle may have a size within a range having any two of the values discussed in this paragraph as endpoints.

In some embodiments, the nanoparticle ranges with diverse morphology comprising sphere, shell, cube, prism, pyramid, star, tube, popcorn, rod, cage, vesicle, or any combination thereof. It is to be appreciated that the morphology of the nanoparticle is not limited to the examples above but can be any suitable morphologies.

In some embodiments, the light source is a red light, a green light, a white light, a blue light, a yellow light or any combinations thereof. The light source can emit the light at a range of predetermined wavelengths for the respective color.

In some embodiments, the irradiating is for a time period between 5 seconds and 30 minutes, between 5 seconds and 15 minutes, between 5 seconds and 10 minutes, between 5 seconds and 5 minutes, between 5 seconds and 2 minutes, between 5 seconds and one minute, between 5 seconds and 30 seconds, between 5 seconds and 15 seconds, between 15 seconds and 30 minutes, between 15 seconds and 15 minutes, between 15 seconds and 10 minutes, between 15 seconds and 5 minutes, between 15 seconds and 2 minutes, between 15 seconds and 60 seconds, between 15 seconds and 30 seconds, between 30 seconds and 30 minutes, between 30 seconds and 15 minutes, between 30 seconds and 10 minutes, between 30 seconds and 5 minutes, between 30 seconds and 2 minutes, between 30 seconds and 60 seconds, between 60 seconds and 30 minutes, between 60 seconds and 15 minutes, between 60 seconds and 10 minutes, between 60 seconds and 5 minutes, between 60 seconds and 2 minutes, between 2 minutes and 30 minutes, between 2 minutes and 15 minutes, between 2 minutes and 10 minutes, between 2 minutes and 5 minutes, between 5 minutes and 30 minutes, between 5 minutes and 15 minutes, between 5 minutes and 10 minutes, between 10 minutes and 15 minutes, between 10 minutes and 30 minutes, or between 15 minutes or 30 minutes. In some embodiments, the irradiating may be for a time period within a range having any two of the values discussed in this paragraph as endpoints.

In some embodiments, the irradiating uses continuous light source. In some embodiments, the irradiating uses Pulse Width Modulation (PWM) to control the brightness of the light source. In some embodiments, the irradiating pulse width duty cycle is 10%, 20%, 30%, 40%, 50%, 60%, 70%, 80%, or 90%. In some embodiments, the irradiating pulse width duty cycle may be any value between 0% and 100%.

In one example, a light source may emit red light to a complex having a target analyte and a gold nanoparticle with a size of 100 nm for at least one minute. In another example, a light source may emit yellow light to a complex having a target analyte and a gold nanoparticle with a size of 100 nm for at least one minute. In still another example, a light source may emit green light to a complex having a target analyte and a gold nanoparticle with a size of 40 nm for at least one minute.

In some embodiments, the complex further comprises a first binding moiety on a substrate and a second binding moiety on the nanoparticle, wherein both the first binding moiety and second binding moiety bind to the target analyte.

As used herein, the term “binding moiety” refers to a molecule, synthetic or natural, that specifically binds or otherwise links to, e.g., covalently or non-covalently binds to or hybridizes with, a target molecule, or with another binding moiety (or, in certain embodiments, with an aggregation inducing molecule). In some embodiments, the binding moiety can be a synthetic oligonucleotide that hybridizes to a specific complementary nucleic acid target. In some embodiments, the binding moiety can be an antibody directed toward an antigen or any protein-protein interaction. In some embodiments, the binding moiety can be a polysaccharide that binds to a corresponding target. In some embodiments, the binding moieties can be designed or selected to serve, when bound to another binding moiety, as substrates for a target molecule such as enzyme in solution. Examples of binding moieties include, but are not limited to, oligonucleotides, polypeptides, antibodies, and polysaccharides. As an example, streptavidin has four sites (binding moieties) per molecule that will be recognized by biotin. For any given analyte, e.g., a specific type of cell having a specific surface marker, there are typically many known binding moieties that are known to those of skill in the relevant fields.

In some embodiments, the substrate is a magnetic bead. In some embodiments, the magnetic bead comprises one or more inner magnetic cores and an outer coating, e.g., a capping polymer. In some embodiments, the magnetic cores can be monometallic (e.g., Fe, Ni, Co), bimetallic (e.g., FePt, SmCo, FePd, FeAu) or can be made of ferrites (e.g., Fe2O3, Fe3O4, MnFe2O4, NiFe2O4, CoFe2O4). In some embodiments, the magnetic bead can be nanometers or micrometers in size, and can be diamagnetic, ferromagnetic, paramagnetic, or superparamagnetic, in which size corresponds to an average diameter or average length. For example, the magnetic bead can have a size of about 10 μm, about 1 μm, about 500 nm, about 300 nm, or about 100 nm. In some embodiments, the magnetic bead having a size of about 2.8 μm can be beneficial in reducing the degree of sedimentation during an assay. Other particle sizes are possible as well. The outer coating of a magnetic bead can increase its water-solubility and stability and also can provide sites for further surface treatment with binding moieties.

In some embodiments, the nanoparticle is conjugated to a reporter enzyme. The reporter enzyme includes, but is not limited to, horseradish peroxidase (HRP), alkaline phosphatase (AP), beta-galactosidase, glucose oxidase (GOx), tyrosinase, urease, a DNAzyme, an aptazyme, or any combination thereof. In some embodiments, the reporter enzyme comprises HRP. It is appreciated that a nanoparticle may not be conjugated to a reporter enzyme according to some embodiments.

In some embodiments, the method comprises introducing an electron mediator into the electrochemical sensor. The electron mediator includes, but is not limited to, 2,2′-Azinobis [3-ethylbenzothiazoline-6-sulfonic acid]-diammonium salt (ABTS), o-phenylenediamine dihydrochloride) (OPD), 3,3′,5,5′-tetramethylbenzidine (TMB), p-nitrophenyl phosphate (PNPP), o-nitrophenyl-β-D-galactopyranoside (ONPG), Naphthol-AS-B1-beta-D-galactopyranosidase (Nap-Gal), 4-Methyl-umbelliferyl-beta-D-galactopyranosidase (MUm-Gal).

In some embodiments, the method further comprises inducing an oxidation-reduction reaction between the electron mediator and the reporter enzyme. Different electron mediator can be used, depending on the choice of a reporter enzyme. For example, when the reporter enzyme comprises HRP, the electron mediator can include water-soluble substrates such as ABTS (2,2′-Azinobis [3-ethylbenzothiazoline-6-sulfonic acid]-diammonium salt), OPD (o-phenylenediamine dihydrochloride), and/or TMB (3,3′,5,5′-tetramethylbenzidine). In another example, when the reporter enzyme comprises alkaline phosphatase, the electron mediator can include water-soluble substrates such as PNPP (p-nitrophenyl phosphate). In another example, when the reactive enzyme includes beta-galactosidase, the electron mediator can include water-soluble substrates such as ONPG (o-nitrophenyl-β-D-galactopyranoside), Nap-Gal (Naphthol-AS-B1-beta-D-galactopyranosidase), and/or MUm-Gal (4-Methyl-umbelliferyl-beta-D-galactopyranosidase).

In some embodiments, the reporter enzyme comprises horseradish peroxidase (HRP), and the electron mediator comprises 3,3′,5,5′-tetramethylbenzidine (TMB).

In some embodiments, the method further comprises inducing an oxidation and reduction reaction between the electron mediator and the reporter enzyme.

In some embodiments, the method comprises including an oxidation and reduction reaction involving hydrogen ions and the nanoparticle through a hydrogen evolution reaction (HER). In some embodiments, the method comprises introducing the hydrogen ions into the electrochemical sensor. In some embodiments, the hydrogen ions are provided in an acid medium including, but not limited to, sulfuric acid, nitric acid, hydrochloric acid, chloric acid, perchloric acid, hydroiodic acid, formic acid, acetic acid, acetic acid, phosphoric acid, hydrofluoric acid, oxalic acid, or any combination thereof, with any suitable concentrations. In some embodiments, the nanoparticle is a metal nanoparticle that can catalyze HER including, but not limited to, gold, silver, platinum, iron, or copper.

In some embodiments, the complex is in proximity to a sample probe of the electrochemical sensor. In some embodiments, the sample probe comprises a screen-printed electrode.

According to one aspect, the present disclosure further provides an amplification method for electrochemical sensing of a target analyte, the method comprising: approximating a complex comprising a nanoparticle and a target analyte to a sample detection region of a first electrode in an electrochemical sensor, wherein the first electrode is electrically coupled to a potentiostat; irradiating the complex with a light source in proximity to the electrochemical senor; and monitoring an output of the potentiostat to determine a presence or quantity of the target analyte.

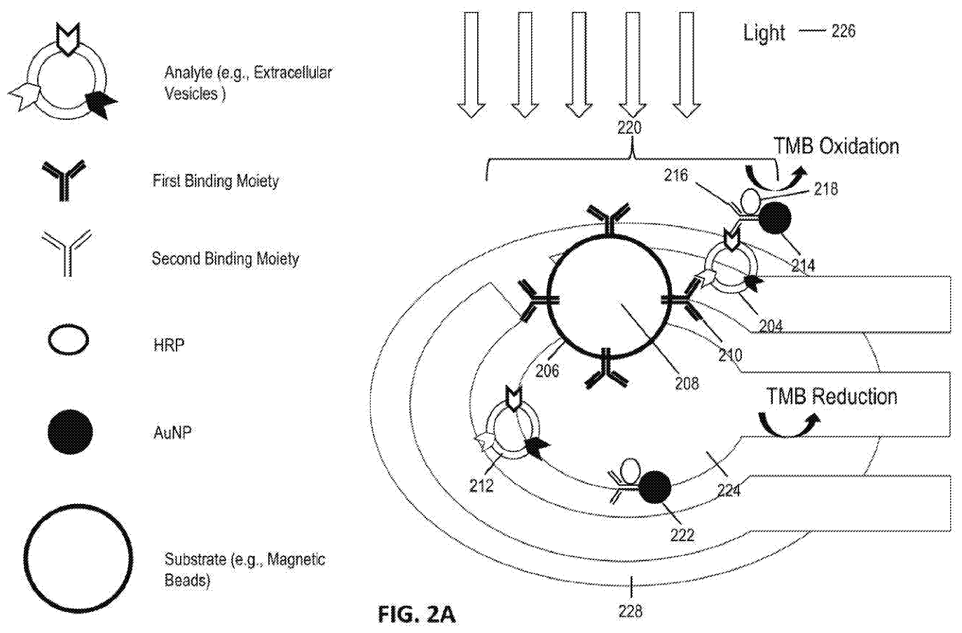

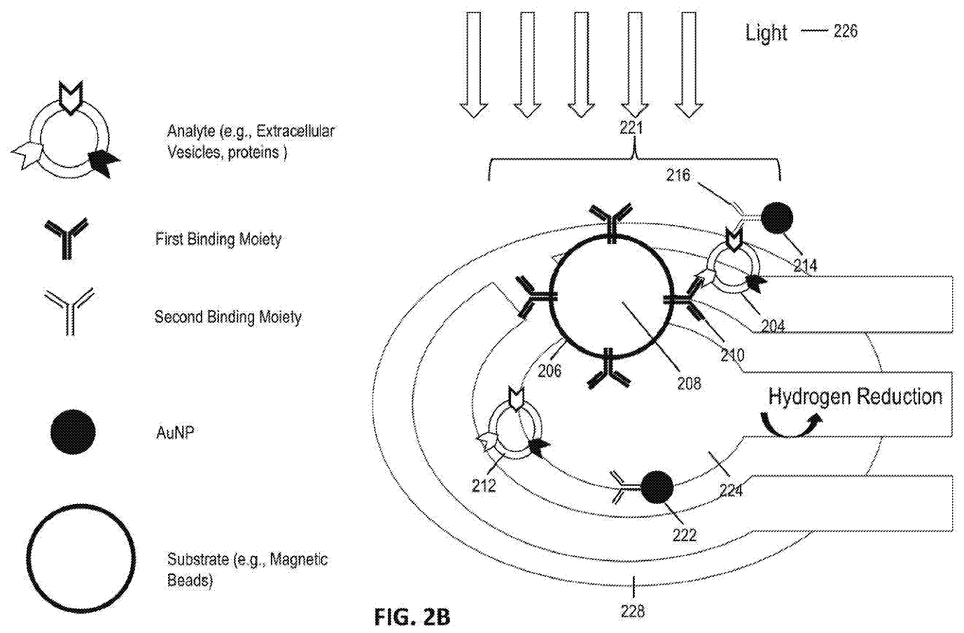

An example usage of the iONE sensing method is shown in FIG. 2A. In this example, the method is used to analyze a fluid sample for the presence or quantity of an analyte based on electron mediator-reporter enzyme reduction-oxidation reaction (redox).

Referring to FIG. 2A, a fluid sample (containing a target analyte 204) is mixed with a solution containing magnetic beads 206. Each magnetic bead 206 comprises a magnetic core 208, and one or more first binding moieties 210 coated onto the surface of magnetic core 208. First binding moieties 210 bind specifically to analyte 204.

Upon mixing, analyte 204 is captured by magnetic bead 206 due to the interaction between first binding moiety 210 and analyte 204. The sample can be washed to remove unbound analytes 212 (e.g., by magnetically collecting magnetic beads 206, and transferring the collected magnetic beads 206 to a fresh sample buffer).

The sample is then mixed with a solution containing a gold nanoparticle 214. Gold nanoparticle 214 comprises a second binding moiety 216 for analyte 204. A reporter enzyme 218 (such as horseradish peroxidase) is conjugated to gold nanoparticle 214. Upon mixing, a complex 220 is formed among gold nanoparticle 214, analyte 204, and magnetic bead 206 due to the interaction between both first binding moiety 210 and second binding moiety 216 and analyte 204. The sample can be washed to remove unbound gold nanoparticles 222. It is understood that other types of nanoparticles, for example, metallic nanoparticles (e.g., platinum nanoparticles, iron nanoparticles, and copper nanoparticles), silica nanoparticles, or organic nanoparticles, may be used in some embodiments.

The sample is then mixed with an electron mediator solution (such as TMB) and is applied to a sample detection region 224 (e.g., including one or more electrodes). In some embodiments, a magnet assembly 228 (e.g., including one or more permanent magnets or electromagnets) may be placed at or in close proximity to sample detection region 224 to concentrate complex 220 at sample detection region 224.

A light source 226 is placed in proximity to sample detection region 224, near complex 220, and the resulting light spot can be similar to the size of working/counter/reference electrodes. Example light sources include laser diode lights and LEDs of various wavelengths. Complex 220 is irradiated by light source 226. The chronoamperometry measurement is taken place with light irradiation using an electrochemical sensor. Example light source and irradiation time include irradiation under a red laser diode (5 mW) for 2 minutes.

In general, the current induced by the electrodes in sample detection region 224 can correlate with the presence and/or concentration of analyte 204 in the sample. For example, if the sample contains a relatively high concentration of analyte 204, a greater number of analytes 204 can be captured by magnetic beads 206. In turn, a greater number of gold nanoparticles 214 can also be captured by magnetic bead 206 and brought into proximity with sample detection region 224. This results in a greater number of reporter enzymes 218 available to react with the electron mediator solution. Accordingly, the oxidation-reduction reaction between reporter enzyme 218 and the electron mediator results in a relatively higher current across the working and counter electrodes. The light irradiation enhances the oxidation-reduction reaction in the presence of nanoparticles, and results in a further enhanced current across the working and counter electrodes.

In contrast, if the sample contains a relatively low concentration of analyte 204, a smaller number of analytes 204 can be captured by magnetic beads 206. In turn, a smaller number of gold nanoparticles 214 can also be captured by magnetic bead 206 and brought into proximity with sample detection region 224. This results in a smaller number of reporter enzymes 218 available to react with the electron mediator solution. The irradiation with light source 226 enhances the oxidation-reduction reaction in the presence of gold nanoparticles 214 and improves the detection limits of analytes 204. Under the same irradiation condition, the enhanced current remains lower compared to the enhanced current generated from high concentration of analytes 204.

Further, if the sample does not contain any analyte 204, substantially no analytes 204 can be captured by magnetic beads 206. In turn, substantially no gold nanoparticles 214 can be captured by magnetic beads 206. Thus, substantially no reporter enzymes 218 can be available to react with the electron mediator solution. Accordingly, substantially no current can be induced across the working and counter electrodes. In this case, the light irradiation does not result in a further enhanced current across the working and counter electrodes.

In some embodiments, the electrochemical sensor can estimate an absolute concentration and/or relative concentration of the analyte based on the induced current. For example, in some implementations, the correlation between induced current and the analyte concentration can be empirically determined (e.g., by obtaining samples having known concentrations of an analyte, measuring the induced current that results from the analysis process, and deriving a function that describes the relationship between the concentration and the induced current). Subsequently, the concentration of an unknown sample can be estimated by calibrating an observed current measurement according to the determined correlation. In some embodiments, the correlation between induced current and the analyte concentration can differ, depending on the analyte, the magnetic beads, the gold nanoparticles, and/or other parameters. Thus, different correlations can be determined for each set of parameters and can be selectively applied as appropriate to interpret a current measurement.

According to one aspect, the present disclosure provides an amplification method for electrochemical sensing of a target analyte, the method comprising: providing a first binding moiety on a substrate to a sample comprising a target analyte, wherein the first binding moiety specifically binds to the target analyte; allowing the target analyte in the sample to bind with the first binding moiety on the substrate, thereby forming a first complex comprising the target analyte and the first binding moiety on the substrate; providing a nanoparticle comprising a second binding moiety binds to the target analyte, and the nanoparticle is conjugated to a reporter enzyme; allowing the first complex to bind with the second binding moiety of the nanoparticle, thereby forming a second complex comprising the first complex and the second binding moiety of the nanoparticle; introducing an electron mediator into the electrochemical sensor; applying the second complex to a sample detection region of a first electrode in the electrochemical sensor, wherein the first electrode is electrically coupled to a potentiostat; irradiating the second complex in the sample detection region with a light source; inducing an oxidation-reduction reaction between the electron mediator and the reporter enzyme; and monitoring an output of the potentiostat to determine a presence or quantity of the target analyte in the sample.

In some embodiments, inducing the oxidation-reduction reaction comprises applying an electrical potential to a second electrode such that the oxidation-reduction reaction occurs, wherein the second electrode is electrically coupled to the potentiostat.

In some embodiments, monitoring the output of the potentiostat comprises measuring a voltage or current from the first electrode, and the voltage or current from the first electrode varies as a result of the oxidation-reduction reaction.

In some embodiments, monitoring the output of the potentiostat comprises: selecting the output of the potentiostat from a plurality of different potentiostats' outputs; providing the selected output to a microcontroller unit; and presenting the selected output on a display.

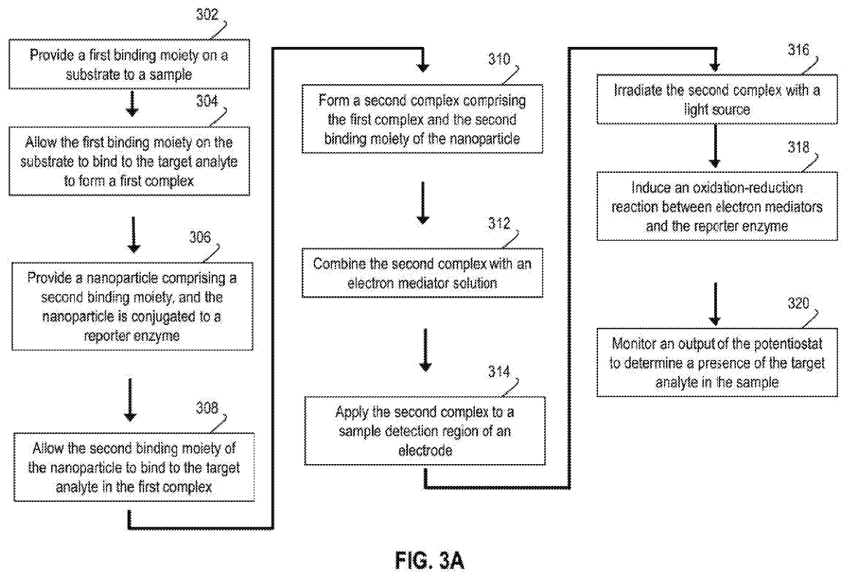

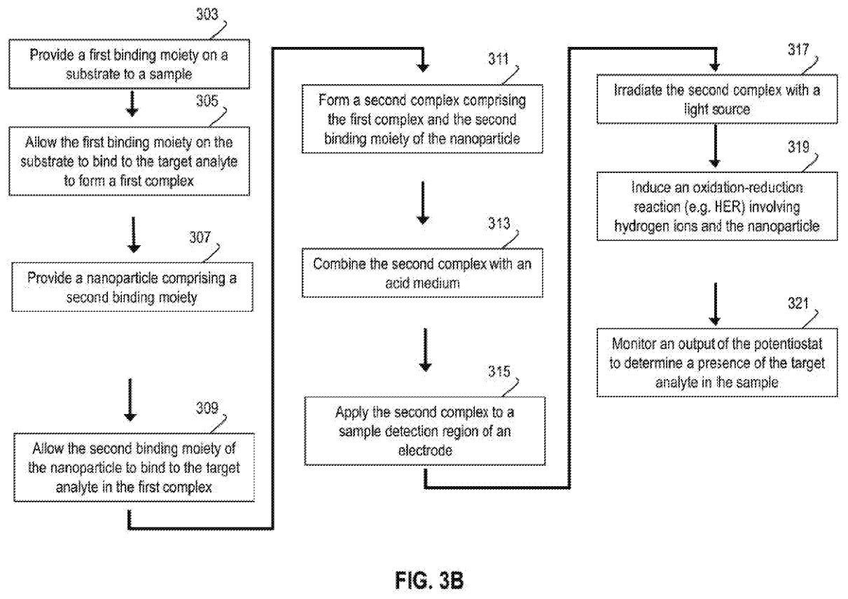

An example process of detecting a presence or quantity of a target analyte in a fluid sample based on light-enhanced electron mediator-reporter enzyme redox is shown in FIG. 3A.

In the process, a first binding moiety on a substrate is provided to a sample in 302. The substrate comprises the first binding moiety specific for binding to the target analyte.

The substrate is allowed to bind to the target analyte through the first binding moiety to form a first complex in 304.

A nanoparticle comprising a second binding moiety for binding to the target analyte is provided to the first complex in 306. The second binding moiety is bound to the nanoparticle and the nanoparticle is conjugated to a reporter enzyme.

The second binding moiety of the nanoparticle is allowed to bind to the target analyte in the first complex in 308.

A second complex comprising the first complex and the second binding moiety of the nanoparticle is formed in 310.

The second complex is combined with an electron mediator solution in 312.

The second complex is provided to a sample detection region of an electrode in 314. The sample detection region is arranged on a first electrode.

The second complex is irradiated with a light source in 316.

An oxidation-reduction reaction between electron mediators is induced within the sample and the reactive enzyme in 318.

A light-triggered electrical current enhancement is induced via the sample, the nanoparticle, the reporter enzyme and the electrode.

An output of the potentiostat is monitored to determine a presence or quantity of the target analyte in the sample in 320. The output of the potentiostat is modified by the oxidation-reduction reaction and the light-induced electrical enhancement.

According to one aspect, the present disclosure also provides an amplification method for electrochemical sensing of a target analyte in a liquid sample, the method comprising: providing a plurality of magnetic beads to a first fluid sample, wherein the plurality of magnetic beads comprises first binding moieties that specifically bind to the target analyte; allowing the first binding moieties of the plurality of magnetic beads to bind to the target analyte in the first fluid sample; transferring the plurality of magnetic beads from the first fluid sample to a second fluid sample, wherein the second fluid sample comprises a plurality of nanoparticles, wherein the plurality of nanoparticles comprise second binding moieties that bind to the target analyte, and the plurality of nanoparticles are conjugated to a plurality of reporter enzymes; allowing the second binding moieties of the plurality of nanoparticles to bind to the target analyte bound to the first binding moieties of the plurality of magnetic beads; combining the seconding fluid sample comprising the plurality of magnetic beads and the plurality of nanoparticles with a solution comprising a plurality of electron mediators to obtain a third fluid sample; providing the third fluid sample to a sample detection region of a first electrode, wherein the first electrode is electrically coupled to a potentiostat; irradiating the third fluid sample with a light source; inducing an oxidation-reduction reaction between the plurality of electron mediators and the reporter enzymes in the third fluid sample; and monitoring an output of the potentiostat to determine a presence or quantity of the target analyte in the third fluid sample.

In some embodiments, transferring the plurality of magnetic beads comprises: immersing a sheath within the first fluid sample; placing a magnet within the sheath such that the plurality of magnetic beads adhere to the sheath; removing the sheath containing the magnet from the first fluid sample; immersing the sheath containing the magnet in the second fluid sample; and removing the magnet from the sheath to release the plurality of magnetic beads to the second fluid sample. It is understood that transfer of the magnetic beads is not limited by the embodiments disclosed herein and can be achieved by any suitable approaches as known in the art.

In some embodiments, the third fluid sample is exposed to a magnetic field to retain the plurality of magnetic beads in the third fluid sample next to the first electrode.

In some embodiments, inducing the oxidation-reduction reaction comprises applying an electrical potential to a second electrode such that the oxidation-reduction reaction occurs, wherein the second electrode is electrically coupled to the potentiostat.

In some embodiments, monitoring the output of the potentiostat comprises measuring a voltage or current from the first electrode, wherein the voltage or current from the first electrode varies as a result of the oxidation-reduction reaction.

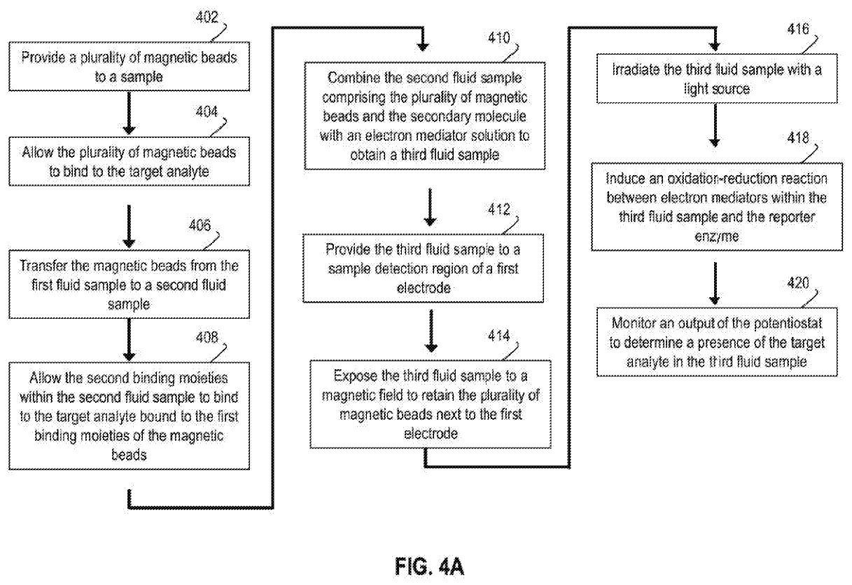

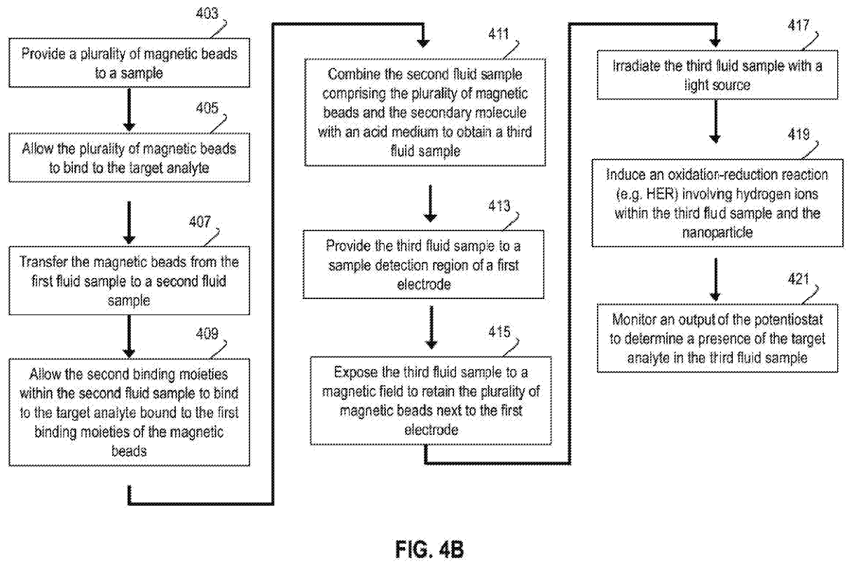

An example process of detecting a presence or quantity of a target analyte in a fluid sample based on light-enhanced electron mediator-reporter enzyme redox is shown in FIG. 4A.

In the process, a plurality of magnetic beads is provided to a first fluid sample in 402. The plurality of magnetic beads includes first binding moieties specific for binding to the target analyte.

The plurality of magnetic beads are allowed to bind to the target analyte within the first fluid sample in 404.

The magnetic beads are transferred from the first fluid sample to a second fluid sample in 406. The second fluid sample includes second binding moieties that are for binding to the target analyte, and the second binding moieties are bound to a nanoparticle and a reporter enzyme.

The second binding moieties within the second fluid sample are allowed to bind to the target analyte bound to the first binding moieties of the magnetic beads in 408.

The second fluid sample including the plurality of magnetic beads and the second binding moieties are combined with an electron mediator solution to obtain a third fluid sample in 410.

The third fluid sample is provided to a sample detection region of an electrode in 412. The sample detection region is arranged on a first electrode.

The third fluid sample is exposed to a magnetic field to retain the plurality of magnetic beads within the third fluid sample next to the first electrode in 414. The first electrode is electrically coupled to a potentiostat.

The third fluid sample is irradiated with a light source in 416.

An oxidation-reduction reaction between electron mediators is induced within the third fluid sample and the reactive enzyme in 418.

A light-triggered electrical current enhancement is induced via the third fluid sample, the nanoparticle, the reporter enzyme and the electrode.

An output of the potentiostat is monitored to determine a presence or quantity of the target analyte in the third fluid sample in 420. The output of the potentiostat is modified by the oxidation-reduction reaction and the light-induced electrical enhancement.

In some embodiments, the process can be performed to identify analyte such as cells, extracellular vesicles such as microvesicles, membrane particles, apoptotic blebs or vesicles, exosomes, proteins, (e.g., secreted, transmembrane and cytosolic proteins, degraded proteins, post-translationally modified proteins, etc.), nucleic acids (e.g. mRNA, DNA, and microRNA, among others), peptides, lipids, metabolites, and other molecules, either free-floating (e.g., in a serum or a solution) or expressed on the surface of a biological structure (e.g., on the surface of an extracellular vesicle or a cell). In some embodiments, the process can be used to provide more effective care to a patient (e.g., by enabling caretakers to make diagnoses and/or administer treatment in a more informed manner). In some embodiments, the process can be performed to provide insight into the composition of the substance (e.g., a food product).

Another example usage of the iONE sensing method is shown in FIG. 2B. In this example, the method is used to analyze a fluid sample for the presence or quantity of an analyte based on HER.

Referring to FIG. 2B, a fluid sample (containing target analyte 204) is mixed with a solution containing magnetic beads 206. Each magnetic bead 206 comprises magnetic core 208, and one or more first binding moieties 210 coated onto the surface of magnetic core 208. First binding moieties 210 bind specifically to analyte 204.

Upon mixing, analyte 204 is captured by magnetic bead 206 due to the interaction between first binding moiety 210 and analyte 204. The sample can be washed to remove unbound analytes 212 (e.g., by magnetically collecting magnetic beads 206, and transferring the collected magnetic beads 206 to a fresh sample buffer).

The sample is then mixed with a solution containing gold nanoparticle 214. Gold nanoparticle 214 comprises second binding moiety 216 for analyte 204. Upon mixing, a complex 221 is formed among gold nanoparticle 214, analyte 204, and magnetic bead 206 due to the interaction between both first binding moiety 210 and second binding moiety 216 and analyte 204. The sample can be washed to remove unbound gold nanoparticles 222.

The sample is then mixed with an acid medium (such as diluted hydrochloric acid) and applied to sample detection region 224 (e.g., including one or more electrodes). In some embodiments, magnet assembly 228 (e.g., including one or more permanent magnets or electromagnets) may be placed at or in close proximity to sample detection region 224 to concentrate complex 220 at sample detection region 224.

Light source 226 is placed in proximity to sample detection region 224, near complex 220, and the resulting light spot can be similar to the size of working/counter/reference electrodes. Example light sources include laser diode lights and LEDs of various wavelengths. Complex 220 is irradiated by light source 226. The chronoamperometry measurement is taken place with light irradiation using an electrochemical sensor. Example light source and irradiation time include irradiation under a red LED for 5 minutes.

In general, the current induced by the electrodes in sample detection region 224 can correlate with the presence and/or concentration of analyte 204 in the sample. For example, if the sample contains a relatively high concentration of analyte 204, a greater number of analytes 204 can be captured by magnetic beads 206. In turn, a greater number of gold nanoparticles 214 can also be captured by magnetic bead 206 and brought into proximity with sample detection region 224. This results in a greater number of gold nanoparticles 214 available to catalyze the hydrogen ions. Accordingly, the oxidation-reduction reaction (e.g., HER) involving the hydrogen ions and gold nanoparticles 214 results in a relatively higher current across the working and counter electrodes. The light irradiation enhances the oxidation-reduction reaction in the presence of nanoparticles, and results in a further enhanced current across the working and counter electrodes.

In contrast, if the sample contains a relatively low concentration of analyte 204, a smaller number of analytes 204 can be captured by magnetic beads 206. In turn, a smaller number of gold nanoparticles 214 can also be captured by magnetic bead 206 and brought into proximity with sample detection region 224. This results in a smaller number of gold nanoparticles 214 available to catalyze the hydrogen ions. The irradiation with light source 226 enhances the oxidation-reduction reaction in the presence of gold nanoparticles 214 and improves the detection limits of analytes 204. Under the same irradiation condition, the enhanced current remains lower compared to the enhanced current generated from high concentration of analytes 204.

Further, if the sample does not contain any analyte 204, substantially no analytes 204 can be captured by magnetic beads 206. In turn, substantially no gold nanoparticles 214 can be captured by magnetic beads 206. Thus, substantially no gold nanoparticles 214 can be available to catalyze the hydrogen ions. Accordingly, substantially no current can be induced across the working and counter electrodes. In this case, the light irradiation does not result in a further enhanced current across the working and counter electrodes.

In some embodiments, the electrochemical sensor can estimate an absolute concentration and/or relative concentration of the analyte based on the induced current. For example, in some implementations, the correlation between induced current and the analyte concentration can be empirically determined (e.g., by obtaining samples having known concentrations of an analyte, measuring the induced current that results from the analysis process, and deriving a function that describes the relationship between the concentration and the induced current). Subsequently, the concentration of an unknown sample can be estimated by calibrating an observed current measurement according to the determined correlation. In some embodiments, the correlation between induced current and the analyte concentration can differ, depending on the analyte, the magnetic beads, the gold nanoparticles, and/or other parameters. Thus, different correlations can be determined for each set of parameters and can be selectively applied as appropriate to interpret a current measurement.

According to one aspect, the present disclosure provides an amplification method for electrochemical sensing of a target analyte, the method comprising: providing a first binding moiety on a substrate to a sample comprising a target analyte, wherein the first binding moiety specifically binds to the target analyte; allowing the target analyte in the sample to bind with the first binding moiety on the substrate, thereby forming a first complex comprising the target analyte and the first binding moiety on the substrate; providing a nanoparticle comprising a second binding moiety that binds to the target analyte; allowing the first complex to bind with the second binding moiety of the nanoparticle, thereby forming a second complex comprising the first complex and the second binding moiety of the nanoparticle; introducing hydrogen ions into the electrochemical sensor; applying the second complex to a sample detection region of a first electrode in the electrochemical sensor, wherein the first electrode is electrically coupled to a potentiostat; irradiating the second complex in the sample detection region with a light source; inducing an oxidation-reduction reaction (e.g. HER) involving the hydrogen ions and the nanoparticle; and monitoring an output of the potentiostat to determine a presence or quantity of the target analyte in the sample.

In some embodiments, inducing the oxidation-reduction reaction comprises applying an electrical potential to a second electrode such that the oxidation-reduction reaction occurs, wherein the second electrode is electrically coupled to the potentiostat.

In some embodiments, monitoring the output of the potentiostat comprises measuring a voltage or current from the first electrode, and the voltage or current from the first electrode varies as a result of the oxidation-reduction reaction.

In some embodiments, monitoring the output of the potentiostat comprises: selecting the output of the potentiostat from a plurality of different potentiostats' outputs; providing the selected output to a microcontroller unit; and presenting the selected output on a display.

An example process of detecting a presence or quantity of a target analyte in a fluid sample based on light-enhanced HER is shown in FIG. 3B.

In the process, a first binding moiety on a substrate is provided to a sample in 303. The substrate comprises the first binding moiety specific for binding to the target analyte.

The substrate is allowed to bind to the target analyte through the first binding moiety to form a first complex in 305.

A nanoparticle comprising a second binding moiety for binding to the target analyte is provided to the first complex in 307. The second binding moiety is bound to the nanoparticle.

The second binding moiety of the nanoparticle is allowed to bind to the target analyte in the first complex in 309.

A second complex comprising the first complex and the second binding moiety of the nanoparticle is formed in 311.

The second complex is combined with an acid medium in 313.

The second complex is provided to a sample detection region of an electrode in 315. The sample detection region is arranged on a first electrode.

The second complex is irradiated with a light source in 317.

An oxidation-reduction reaction (e.g. HER) involving hydrogen ions and the nanoparticle is induced within the sample in 319.

A light-triggered electrical current enhancement is induced via the sample, the nanoparticle, the hydron ions, and the electrode.

An output of the potentiostat is monitored to determine a presence or quantity of the target analyte in the sample in 321. The output of the potentiostat is modified by the oxidation-reduction reaction and the light-induced electrical enhancement.

According to one aspect, the present disclosure also provides an amplification method for electrochemical sensing of a target analyte in a liquid sample, the method comprising: providing a plurality of magnetic beads to a first fluid sample, wherein the plurality of magnetic beads comprises first binding moieties that specifically bind to the target analyte; allowing the first binding moieties of the plurality of magnetic beads to bind to the target analyte in the first fluid sample; transferring the plurality of magnetic beads from the first fluid sample to a second fluid sample, wherein the second fluid sample comprises a plurality of nanoparticles, wherein the plurality of nanoparticles comprise second binding moieties that bind to the target analyte; allowing the second binding moieties of the plurality of nanoparticles to bind to the target analyte bound to the first binding moieties of the plurality of magnetic beads; combining the seconding fluid sample comprising the plurality of magnetic beads and the plurality of nanoparticles with an acid medium solution to obtain a third fluid sample; providing the third fluid sample to a sample detection region of a first electrode, wherein the first electrode is electrically coupled to a potentiostat; irradiating the third fluid sample with a light source; inducing an oxidation-reduction reaction (e.g. HER) involving hydrogen ions and the nanoparticles in the third fluid sample; and monitoring an output of the potentiostat to determine a presence or quantity of the target analyte in the third fluid sample.

In some embodiments, transferring the plurality of magnetic beads comprises: immersing a sheath within the first fluid sample; placing a magnet within the sheath such that the plurality of magnetic beads adhere to the sheath; removing the sheath containing the magnet from the first fluid sample; immersing the sheath containing the magnet in the second fluid sample; and removing the magnet from the sheath to release the plurality of magnetic beads to the second fluid sample. It is understood that transfer of the magnetic beads is not limited by the embodiments disclosed herein and can be achieved by any suitable approaches as known in the art.

In some embodiments, the third fluid sample is exposed to a magnetic field to retain the plurality of magnetic beads in the third fluid sample next to the first electrode.

In some embodiments, inducing the oxidation-reduction reaction comprises applying an electrical potential to a second electrode such that the oxidation-reduction reaction occurs, wherein the second electrode is electrically coupled to the potentiostat.

In some embodiments, monitoring the output of the potentiostat comprises measuring a voltage or current from the first electrode, wherein the voltage or current from the first electrode varies as a result of the oxidation-reduction reaction.

An example process of detecting a presence or quantity of a target analyte in a fluid sample based on light-enhanced HER is shown in FIG. 4B.

In the process, a plurality of magnetic beads is provided to a first fluid sample in 403. The plurality of magnetic beads includes first binding moieties specific for binding to the target analyte.

The plurality of magnetic beads are allowed to bind to the target analyte within the first fluid sample in 405.

The magnetic beads are transferred from the first fluid sample to a second fluid sample in 407. The second fluid sample includes second binding moieties that are for binding to the target analyte, and the second binding moieties are bound to a nanoparticle.

The second binding moieties within the second fluid sample are allowed to bind to the target analyte bound to the first binding moieties of the magnetic beads in 409.

The second fluid sample including the plurality of magnetic beads and the second binding moieties are combined with an acid medium to obtain a third fluid sample in 411.

The third fluid sample is provided to a sample detection region of an electrode in 413. The sample detection region is arranged on a first electrode.

The third fluid sample is exposed to a magnetic field to retain the plurality of magnetic beads within the third fluid sample next to the first electrode in 415. The first electrode is electrically coupled to a potentiostat.

The third fluid sample is irradiated with a light source in 417.

An oxidation-reduction reaction (e.g. HER) involving hydrogen ions and the nanoparticles within the third fluid sample is induced in 419.

A light-triggered electrical current enhancement is induced via the third fluid sample, the nanoparticles, the hydrogen ions, and the electrode.

An output of the potentiostat is monitored to determine a presence or quantity of the target analyte in the third fluid sample in 421. The output of the potentiostat is modified by the oxidation-reduction reaction and the light-induced electrical enhancement.

Applications of the iONE Sensing Method

The implementations described herein can be used in a variety of different applications, including the detection and quantification of various peptides, proteins, lipids metabolites, and other molecules, either free-floating (e.g., in a serum or a solution) or expressed on the surface of a biological structure (e.g., on the surface of an extracellular vesicle or a cell). In some embodiments, the detection and quantification of molecules can provide insight into a particular biological or pathogenic process, a particular progression of a particular disease, or some other biological condition.

Examples of various applications are discussed in more detail below and in the Examples section.

Extracellular Vesicle Screening—Cancer Diagnostics

As an example, iONE sensing can be used to diagnosing cancer through extracellular vesicles found in blood or other body fluids such as urine.

Growing evidence has positioned EVs as an effective readout of cancer management. Extracellular vesicles (EV, including exosomes) for example, have emerged as a potent biomarker. Exosomes are nanoscale vesicles actively secreted by cells. These vesicles carry molecular constituents of their originating cells, including transmembrane and cytosolic proteins, mRNA, DNA, and microRNA, and can thus serve as cellular surrogates. Combined with their relative abundance and ubiquitous presence in bodily fluids (e.g. serum, ascites, urine, CSF), exosomes can offer unique advantages for longitudinal monitoring. Exosome analyses are minimally invasive and afford relatively unbiased readouts of the entire tumor burden, less affected by the scarcity of the samples or intratumoral heterogeneity.

iONE sensing in combination with magnetic capture can be an effective detection modality that is easily applicable to clinical settings. It could achieve high sensitivity through signal amplification with redox-active reporters or HER. As described herein, a sensing system can measure electrical currents induced by the redox-active reporters or HER and can be realized as a compact and low-power portable device.

As described herein, implementations of the sensing system provide various benefits. For example, the assay can achieve high detection sensitivity through magnetic enrichment, enzymatic amplification or HER, and opto nano signal enhancement.

As an example, ovarian cancer exosomes are often enriched with EpCAM and CD24. Thus, implementations of the sensing system can be used to profile EpCAM, CD24, or both expressing EV populations (e.g., exosomes) as a means of diagnosing ovarian cancer.

For instance, magnetic beads can be conjugated to antibodies specific against EpCAM, CD24, or both. These magnetic beads can be mixed with a biological sample containing exosomes (e.g., plasma), such that exosomes expressing EpCAM, CD24, or both can be magnetically captured. In turn, the sample can be treated with secondary molecules specific to exosomes expressing EpCAM, CD24, or both. The second molecule includes a labeling ligand (such as antibodies specific against expressing EpCAM or CD24), which is conjugated to a gold nanoparticle (AuNP) with or without an oxidizing enzyme (e.g., a streptavidin-HRP). The sample can then be combined with either an electron mediator solution (e.g., a solution containing 3,3′,5,5′-tetramethylbenzidine, TMB) or acidic media.

The sample can be subsequently analyzed using the iONE sensing systems described herein. As the EpCAM, CD24, or both expressing exosomes have been captured by the magnetic beads, they are concentrated near the electrodes of the sensing system. Further, due to the potential induced across the electrodes (e.g., the working electrode and the reference electrode), an oxidation-reduction reaction is induced. As a result, a current is induced across one of the electrodes (e.g., the counter electrode), correlating with the presence and prevalence of expressing EpCAM, CD24, or both expressing exosomes. In turn, this current can be measured by an electrochemical detecting device, and the resulting information can be used for investigative or diagnostic purposes. For example, a relatively high current can correspond to a relatively high concentration of EpCAM, CD24, or both expressing exosomes, and may be an indicator of ovarian cancer in a patient.

In some embodiments, the output of an electrochemical detecting device (such as a potentiostat) can be compared to a threshold or reference level, and the presence or absence of a cancer within a patient can be diagnosed based on the comparison. For example, if the output of a potentiostat is sufficiently high (e.g., a current that exceeds a reference or threshold level), a diagnosis regarding the presence of a cancer can be rendered. However, if the output of the potentiostat is relatively low (e.g., a current that does not exceed the reference or threshold level), a diagnosis regarding the absence of a cancer can be rendered. In some embodiments, an instrument can render a diagnosis automatically or semi-automatically based on the measurements.

Although the detection of exosomes expressing EpCAM or CD24 in blood is described above, this is merely an example. In practice, implementations of the sensing system can be used to detect any biomarker (e.g., biomarkers associated with different biological or pathogenic processes, diseases, or other biological conditions), either free-floating (e.g., free-floating in plasma, urine, or any other biological sample) or expressed on the surface of a biological structure (e.g., on the surface of an extracellular vesicle or a cell). As an example, in some implementations, the sensing system can be used to detect one or more of the following biomarkers: EPCAM, EGFR, HER2, MUC1, MUC2, MUC6, MUC5AC, GPC1, WNT2, CEP, CD24, GPA33, CA125, CD44, CD44v6, CEA, Mesothelin, Trop2, Grp94, SSTR2, CD166, CD133, MET, B7H3, CD63, CD9, CD81, CD2, CD3, CD14, CD45, CD47, CD52, CD68, CD73 HLA-ABC, CXCL10, CXCL9, HVEM, FGFR3, NUMA, HSP70, IL-3, TSP2, PD-L1, EGFRv3, EGFR T790M, IDH1 mutant, APC mutant, KRAS mutant, BRAF mutant, PIK3CA mutant, BRAC1/2 mutant, SMAD4 mutant, CDKN2 mutant, and PTEN mutant among others, which may be useful in diagnosing cancer or assessing immunotherapies.

Extracellular Vesicle Screening—Organ Rejection Testing

As another example, iONE sensing can be used to detect transplanted organ rejection in a patient.

For instance, in patients experiencing transplanted kidney rejection, CD2, CD3, HLA-ABC, CD52 expressing EVs are often found in the patient's urine. Thus, implementations of the sensing system can be used to profile CD2, CD3, HLA-ABC, CD52 expressing EV populations (e.g., exosomes) as a means of detecting transplanted kidney rejection at the early stages of rejection.

For instance, magnetic beads can be conjugated to a cocktail of antibodies specific against CD2, CD3, HLA-ABC, CD52. These magnetic beads can be mixed with a biological sample containing exosomes (e.g., urine), such that exosomes expressing CD2, CD3, HLA-ABC, CD52 can be magnetically captured. In turn, the sample can be treated with secondary molecules specific to CD2, CD3, HLA-ABC, or CD52 expressing exosomes. The second molecule includes a labeling ligand (such as antibodies specific against CD2, CD3, HLA-ABC, CD52), which is conjugated to a gold nanoparticle (AuNP) with or without an oxidizing enzyme (e.g., a streptavidin-HRP). The sample can then be combined with either an electron mediator solution (e.g., a solution containing 3,3′,5,5′-tetramethylbenzidine, TMB) or acidic media.

The sample can be subsequently analyzed using the sensing systems described herein, and the resulting information can be used for investigative or diagnostic purposes. For example, a relatively high current can correspond to a relatively high concentration of CD2, CD3, HLA-ABC, CD52 expressing exosomes, and may be an indicator of kidney rejection by a patient. In some cases, a patient can be regularly screened after a kidney transplant, such that a kidney rejection is quickly detected at an early stage. As the monitoring process is non-invasive, the monitoring process can be repeated with minimal risk to the patient and in a cost-effective manner.

In some embodiments, the output of a potentiostat can be compared to a threshold or reference level, and a determination regarding whether or not a patient has rejected an organ transplant can be diagnosed based on the comparison. For example, if the output of a potentiostat is sufficiently high (e.g., a current that exceeds a reference or threshold level), a determination that the patient has rejected the organ can be made. However, if the output of the potentiostat is relatively low (e.g., a current that does not exceed the reference or threshold level), a determination that the patient has not rejected the organ can be made. In some cases, an instrument can make a determination automatically or semi-automatically based on the measurements.

Although the detection of exosomes expressing CD2, CD3, HLA-ABC, CD52 in urine is described above, this is merely an illustrate example. As described herein, implementations of the sensing system can be used to detect any biomarker, either free-floating or expressed on the surface of a biological structure, to probe transplanted kidney rejection, or the rejection of other transplanted organs.

### EXAMPLES

The following examples are illustrative and non-limiting, of the products and methods described herein. Suitable modifications and adaptations of the variety of conditions, formulations, and other parameters normally encountered in the field and which are obvious to those skilled in the art in view of this disclosure are within the spirit and scope of the present disclosure.

The purpose of this example was to demonstrate the use of iONE sensing to amplify the electrical current generated from oxidation-reduction reaction (e.g., with redox-active reporters or HER).

Conjugation of Gold Nanoparticles(AuNP) with Biotinylated Horseradish Peroxidase

To prepare AuNP-HRP conjugates, 1 OD of the Neutravidin-coated spherical gold nanoparticles (100 nm, Nanopartz) were mixed with 100 μL of biotinylated horseradish peroxidase (100 ug/mL, Rockland) in PBS buffer, and incubated at room temperature for 1 hour. The resultant AuNP-HRP conjugate mixture were kept on ice and brought to room temperature before dilution and use.

Conjugation of Gold Nanoparticles (AuNP) with Biotinylated Anti-IgG

To prepare 100 nm AuNP-IgG conjugates, 4.5 OD of the Neutravidin-coated 100 nm spherical gold nanoparticles (Nanopartz) were mixed with 7 microgram of biotinylated anti-IgG antibody (R&D systems) in PBS buffer, and incubated at room temperature for 30 min. The AuNP-IgG conjugates were blocked using PBS (1% BSA) for 25 min at room temperature and washed twice with 1% PBS (0.05% Tween 20). After washes, the AuNP-IgG conjugates were re-suspended in PBS (0.05% Tween 20) and kept at 4° C. until use.

To prepare 40 nm AuNP-IgG conjugates, 4 OD of the Neutravidin-coated 40 nm spherical gold nanoparticles (Nanopartz) were mixed with 7 microgram of biotinylated anti-IgG antibody (R&D systems) in PBS buffer, and incubated at room temperature for 30 min. The AuNP-IgG conjugates were blocked using PBS (1% BSA) for 25 min at room temperature and washed twice with 1% PBS (0.05% Tween 20). After washes, the AuNP-IgG conjugates were re-suspended in PBS (0.05% Tween 20) and kept at 4° C. until use.

Preparation of Immunomagnetic Beads

Five milligrams of magnetic beads coated with epoxy groups (Dynabeads M-270 Epoxy, Invitrogen) was suspended in 1 mL of 0.1 M sodium phosphate solution at room temperature for 10 min. The magnetic beads were separated from the solution with a permanent magnet and resuspended in 100 μL of the same solution. One hundred micrograms of antibodies against CD63 (Ancell), HVEM (R&D systems) or respective IgG (Biolegend) was added and mixed thoroughly. One hundred microliters of 3 M ammonium sulfate solution were added, and the whole mixture was incubated overnight at 4° C. with slow tilt rotation. The beads were washed twice with PBS solution and finally resuspended in 1 mL of PBS with 1% bovine serum albumin (BSA).

Biotinylation of Labeling Antibodies

Sulfo-NHS-biotin (10 mM, Pierce) solution in PBS was incubated with antibodies for 1 hour at room temperature. Unreacted sulfo-NHS-biotin was removed using Zeba spin desalting column, 7K MWCO (Thermo Scientific). Antibodies were kept at 4° C. until use.

Description of the Electrochemical Device

The detection device included of a micro-controller (Atmel Corporation; or Texas Instruments), a digital-to-analog converter (Texas Instruments), an analog-to-digital converter (Texas Instruments), a multiplexer (Analog Devices), and eight potentiostats. Each potentiostat included of two operational amplifiers (Analog Devices): one amplifier maintains the potential difference between a working electrode and a reference electrode, and the other one works as a transimpedance amplifier to convert a current to a voltage signal. The gold and carbon electrodes are commercially available (DropSens, Spain).

### Example 1: iONE Assay (TMB)—AuNP-HRP Detection

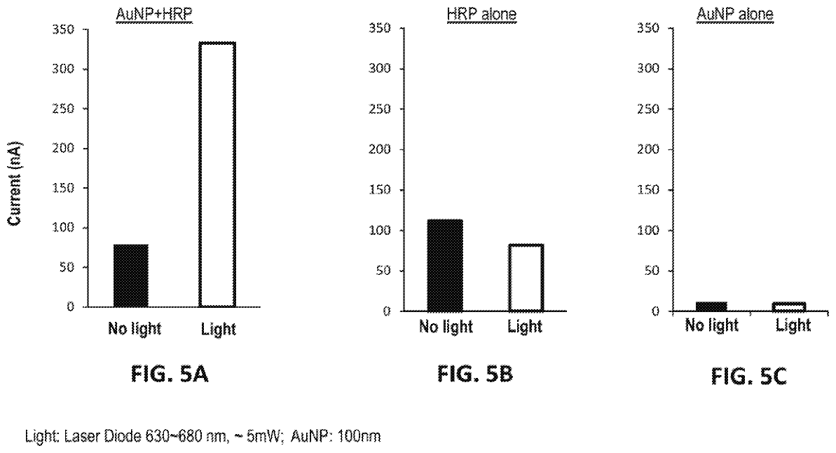

50 μL of UltraTMB solution (Thermo-Fisher Scientific) was loaded on top of the screen-printed gold electrode (DropSens) and then mixed with 10 μL of the diluted AuNP-HRP conjugate. The reaction mixture could fully cover the surface of the working/counter/reference electrodes. The light source (red laser diode light about 5 mW) was placed on top of the electrode, near the reaction mixture (<2 inch), and the resulting light spot was similar to the size of the working/counter/reference electrodes. The reaction mixture was irradiated under red light for one minute and chronoamperometry measurement was started with an electrochemical sensor, with the red light still on. The working electrode potential (−100 mV) was applied and the current levels in the range of 50-60 seconds was averaged (Ilight). In a control assay without light irradiation, 50 μL of UltraTMB solution (Thermo-Fisher Scientific) was loaded on top of a screen-printed gold electrode (DropSens) and then mixed with 10 μL of the conjugated AuNP-HRP. After one minute, chronoamperometry measurement was started with an electrochemical sensor. The working electrode potential (−100 mV) was applied and the current levels in the range of 50-60 seconds was averaged (Ictrl). Significant enhancement of I_{light} compared with I_{ctrl} was observed as shown in FIG. 5A.

Additional control experiments were performed to determine if light-induced current enhancement could be produced in the absence of the reaction enzyme (HRP) or nanoparticles (AuNP). In one control experiment, 50 μL of UltraTMB solution (Thermo-Fisher Scientific) was loaded on top of the screen-printed gold electrode (DropSens) and then mixed with 10 μL of the biotinylated horseradish peroxidase (100 pg/mL, Rockland). The reaction mixture could fully cover the surface of the working/counter/reference electrodes. The light source (red laser diode light about 5 mW) was placed on top of the electrode, near the reaction mixture (<2 inch), and the resulting light spot was similar to the size of the working/counter/reference electrodes. The reaction mixture was irradiated under red light for one minute and chronoamperometry measurement was started with an electrochemical sensor, with the red light still on. The working electrode potential (−100 mV) was applied and the current levels in the range of 50-60 s was averaged (I_{HRP}). In another control experiment, 50 μL of UltraTMB solution (Thermo-Fisher Scientific) was loaded on top of the screen-printed gold electrode (DropSens) and then mixed with 10 μL of the AuNP (100 nm, 5 OD/mL, Nanopartz). The reaction mixture could fully cover the surface of the working/counter/reference electrodes. The light source (red laser diode light ˜5 mW) was placed on top of the electrode, near the reaction mixture (<2 inch), and the resulting light spot was similar to the size of the working/counter/reference electrodes. The reaction mixture was irradiated under red light for one minute and chronoamperometry measurement was started with an electrochemical sensor, with the red light still on. The working electrode potential (−100 mV) was applied and the current levels in the range of 50-60 s was averaged (I_{AuNP}). No significant current enhancement in I_{HRP} or I_{AuNP} under light irradiation was observed as shown in FIGS. 5B and 5C.

This example demonstrated the use of iONE sensing to amplify the electrical current generated from TMB oxidation-reduction reaction.

### Example 2: iONE Assay (HER)—AuNP Detection

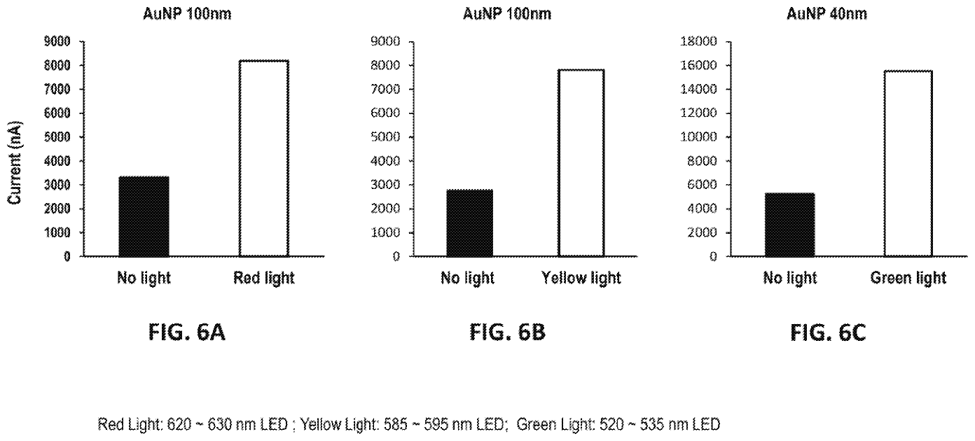

25 μL of the diluted Neutravidin-coated spherical gold nanoparticles (Nanopartz) was loaded on top of the screen-printed carbon electrode (DropSens) and then mixed with 25 μL of 0.1 N HCl solution (Sigma). The reaction mixture could fully cover the surface of the working/counter/reference electrodes. The light source (red LED 620˜630 nm, yellow LED 585˜595 nm, or green LED 520˜535 nm) was placed on top of the electrode, near the reaction mixture (<1 inch), and the working/counter/reference electrodes were fully under light illumination. The reaction mixture was irradiated under red light (or yellow light) for one minute at a potential of 1.35 V and chronoamperometry measurement was started with an electrochemical sensor, with the light still on. The working electrode potential (−1.0 V) was applied and the current levels in the range of 290-300 seconds were averaged (I_{light}). In a control assay without light irradiation, 25 μL of the diluted 100 nm Neutravidin-coated spherical gold nanoparticles (Nanopartz) was loaded on top of the screen-printed carbon electrode (DropSens) and then mixed with 25 μL of 0.1N HCl solution (Sigma). After one minute at the potential of 1.35 V, chronoamperometry measurement was started with an electrochemical sensor. The working electrode potential (−1.0 V) was applied and the current levels in the range of 290-300 seconds was averaged (I_{no-light}). Significant enhancement of I_{light} compared with I_{no-light} was observed as shown in FIG. 6A-C. The detection of AuNP was improved due to light irradiation.

### Example 3: iONE Assay—Urine Protein HVEM Detection

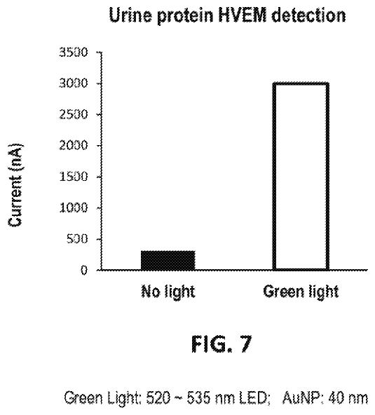

Twenty (20) μL of diluted urine sample from healthy donor was mixed with 25 μL of HVEM-magnetic beads (or control beads) solution and incubated for 15 min at room temperature. 5 μL of biotinylated anti-HVEM (20 μg/mL in PBS) was then mixed with the beads for 15 min at room temperature. The beads were separated from the solution with a permanent magnet and resuspended in 50 μL of PBS (1% BSA, 0.05% Tween 20). 5 μL of 40 nm AuNP-IgG conjugates were mixed with the beads for 15 min at room temperature. For washing, the beads were separated from the solution with a permanent magnet and resuspended in 100 μL of PBS (1% BSA, 0.05% Tween 20). After 5 seconds of vortexing, the beads were separated and resuspended in 25 μL of PBS. These bead-protein-AuNP mixture was loaded on top of the screen-printed carbon electrode (DropSens) and then mixed with 25 μL of 0.1N HCl solution (Sigma). The reaction mixture could fully cover the surface of the working/counter/reference electrodes. Small cylindrical magnets were located below the electrodes to concentrate the magnetic beads to the surface. The light source (green LED 520˜535 nm) was placed on top of the electrode, near the reaction mixture (<1 inch), and the working/counter/reference electrodes were fully under light illumination. The reaction mixture was irradiated under green light for one minute at a potential of 1.35 V and chronoamperometry measurement was started with an electrochemical sensor, with the light still on. The working electrode potential (−1.0 V) was applied and the current levels in the range of 290-300 s were averaged (I_{light HVEM}, or I_{light ctrl}). Δ I_{light} was calculated using the equation: Δ I_{light}=I_{light HVEM}−I_{light ctrl}. In a control assay without light irradiation, 25 μL of the bead-HVEM-AuNP mixture was loaded on top of the screen-printed carbon electrode (DropSens) and then mixed with 25 μL of 0.1N HCl solution (Sigma). After one minute at the potential of 1.35 V, chronoamperometry measurement was started with an electrochemical sensor. The working electrode potential (−1.0 V) was applied and the current levels in the range of 290-300 seconds were averaged (I_{no-light HVEM}, or I_{no-light ctrl}). Δ I_{no-light} was calculated using the equation: ΔI_{no-light}=I_{no-light HVEM}−I_{no-light ctrl}. Significant enhancement of Δ I_{light} compared with ΔI_{no-light} was observed as shown in FIG. 7. The detection of HVEM expression in urine was improved due to light irradiation.

### Example 4: iONE Assay—Tumor Extracellular Vesicle (EV) Protein Detection

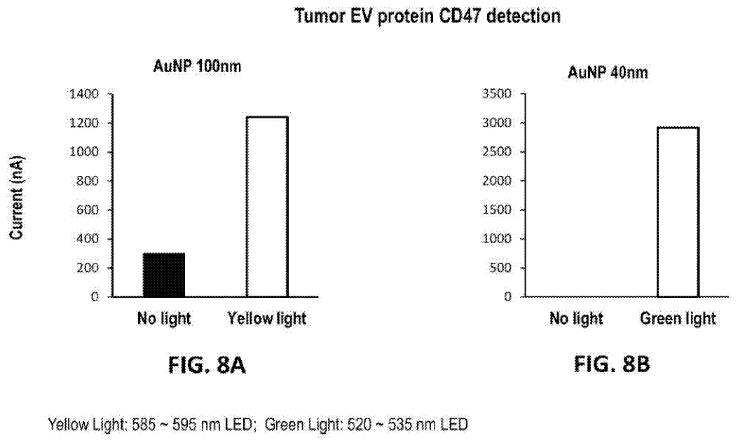

Bladder cancer cells (ATCC) at passages 1-15 were cultured in vesicle-depleted medium for 24˜48 hours. Conditioned medium from approximately 10⁶ cells was collected and centrifuged at 400 g for 10 min. 80 μL of the supernatant was mixed with 40 μL of CD63-magnetic beads (or control beads) solution and incubated for 15 min at room temperature. 5 μL of biotinylated anti-CD47 (40 μg/mL in PBS) was then mixed with the beads for 15 min at room temperature. The beads were separated from the solution with a permanent magnet and resuspended in 50 μL of PBS (1% BSA, 0.05% Tween 20). 5 μL of AuNP-IgG conjugates were mixed with the beads for 15 min at room temperature. For washing, the beads were separated from the solution with a permanent magnet and resuspended in 100 μL of PBS (1% BSA, 0.05% Tween 20). After 5 seconds of vortexing, the beads were separated and resuspended in 25 μL of PBS. These bead-EV CD47-AuNP mixture was loaded on top of the screen-printed carbon electrode (DropSens) and then mixed with 25 μL of 0.1N HCl solution (Sigma). The reaction mixture could fully cover the surface of the working/counter/reference electrodes. Small cylindrical magnets were located below the electrodes to concentrate the magnetic beads to the surface. The light source was placed on top of the electrode, near the reaction mixture (<1 inch), and the working/counter/reference electrodes were fully under light illumination. The reaction mixture was irradiated under light for one minute at a potential of 1.35 V and chronoamperometry measurement was started with an electrochemical sensor, with the light still on. The working electrode potential (−1.0 V) was applied and the current levels in the range of 290-300 seconds were averaged (I_{light CD47}, or I_{light ctrl}). ΔI_{light} was calculated using the equation: Δ I_{light}=I_{light CD47}−I_{light ctrl}. In a control assay without light irradiation, 25 μL of the bead-EV CD47-AuNP mixture was loaded on top of the screen-printed carbon electrode (DropSens) and then mixed with 25 μL of 0.1N HCl solution (Sigma). After one minute at the potential of 1.35 V, chronoamperometry measurement was started with an electrochemical sensor. The working electrode potential (−1.0 V) was applied and the current levels in the range of 290-300 seconds were averaged (I_{no-light CD47}, or I_{no-light ctrl}). ΔI_{no-light} was calculated using the equation: ΔI_{no-light}=I_{no-light CD47}−I_{no-light ctrl}. Significant enhancement of ΔI_{light} compared with Δ I_{no-light} was observed as shown in FIGS. 8A and 8B. The detection of CD47 expression in EV was improved due to light irradiation.

A unique feature of the sensing method is the integration of nanoparticles and light irradiation into a simple platform. It could be easily combined with magnetic capture, nucleic acid hybridization, and electrochemical reaction, to improve the limit of detection for a variety of target analytes. The iONE assay could be a powerful clinical tool for affordable, scalable, and comprehensive EV analyses, thereby deepening our insights into tumor biology and accelerating effective cancer management.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure or the appended claims in any way.

While the present disclosure has been described herein with reference to exemplary embodiments for exemplary fields and applications, it should be understood that the present disclosure is not limited thereto. Other embodiments and modifications thereto are possible, and are within the scope and spirit of the present disclosure. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments may perform functional blocks, steps, operations, methods, etc. using orderings different than those described herein.

The breadth and scope of the present disclosure should not be limited by any of the above-described exemplary embodiments, but should be defined only in accordance with the following claims and their equivalents.

All publications, patents and patent applications mentioned in this specification are indicative of the level of skill of those skilled in the art to which the present disclosure pertains, and are herein incorporated by reference to the same extent as if each individual publication, patent or patent application was specifically and individually indicated to be incorporated by reference.

What is claimed is:

1. A method of signal amplification coupled with detection of a target analyte in a sample, the method comprising:
providing a first binding moiety on a substrate to a sample comprising a target analyte, wherein the first binding moiety specifically binds to the target analyte;
allowing the target analyte in the sample to bind with the first binding moiety on the substrate, thereby forming a first complex comprising the target analyte and the first binding moiety on the substrate;
providing a nanoparticle comprising a second binding moiety that binds to the target analyte;
allowing the first complex to bind with the second binding moiety of the nanoparticle, thereby forming a second complex comprising the first complex and the second binding moiety of the nanoparticle;
introducing hydrogen ions into the electrochemical sensor;
applying the second complex to a sample detection region of a first electrode, wherein the first electrode is electrically coupled to a potentiostat;
irradiating the second complex in the sample detection region with a light source to modify reactions;
inducing an oxidation-reduction reaction involving the hydrogen ions and the nanoparticle; and
monitoring an output of the potentiostat to determine a presence or quantity of the target analyte in the sample.

2. The method of claim 1, wherein inducing the oxidation-reduction reaction comprises applying an electrical potential to a second electrode such that the oxidation-reduction reaction occurs, wherein the second electrode is electrically coupled to the potentiostat.

3. The method of claim 1, wherein
monitoring the output of the potentiostat comprises measuring a voltage or current from the first electrode, and
the voltage or current from the first electrode varies as a result of the oxidation-reduction reaction.

4. The method of claim 1, wherein monitoring the output of the potentiostat comprises:
selecting the output of the potentiostat from a plurality of different potentiostats' outputs;
providing the selected output to a microcontroller unit; and
presenting the selected output on a display.

5. The method of claim 1, wherein the hydrogen ions are in an acid medium.

6. The method of claim 1, wherein the target analyte comprises extracellular vesicles selected from the group consisting of exosomes, microvesicles, oncosomes, apoptotic bodies, and any combination thereof.

7. The method of claim 1, wherein the target analyte comprises a protein, a cell, a peptide, a protein, a lipid, a toxin, nucleic acids, microbes, food antigens, or a metabolite.

8. The method of claim 1, wherein the target analyte comprises at least one biomarker selected from the group consisting of EPCAM, EGFR, HER2, MUC1, MUC2, MUC6, MUC5AC, GPC1, WNT2, CEP, CD24, GPA33, CA125, CD44, CD44v6, CEA, Mesothelin, Trop2, Grp94, SSTR2, CD166, CD133, MET, B7H3, CD63, CD9, CD81, CD2, CD3, CD14, CD45, CD47, CD52, CD68, CD73 HLA-ABC, CXCL10, CXCL9, HVEM, FGFR3, NUMA, HSP70, IL-3, TSP2, PD-L1, EGFRv3, EGFR T790M, IDH1 mutant, APC mutant, KRAS mutant, BRAF mutant, PIK3CA mutant, BRAC1/2 mutant, SMAD4 mutant, CDKN2 mutant, and PTEN mutant biomarkers.

9. The method of claim 1, wherein the nanoparticle comprises at least one metal selected from the group consisting of gold, silver, platinum, iron and copper.

10. The method of claim 1, wherein the nanoparticle ranges in size from about 1 nm to about 1000 nm.

11. The method of claim 1, wherein a morphology of the nanoparticle comprises sphere, shell, cube, prism, pyramid, star, tube, popcorn, rod, cage, vesicle, or any combination thereof.

12. The method of claim 1, wherein the light source is a red light, a green light, a white light, a blue light, a yellow light or any combinations thereof.

13. The method of claim 1, wherein the irradiating is for a time between about 5 seconds and about 5 minutes.

14. The method of claim 1, wherein the substrate comprises a plurality of magnetic beads.

15. The method of claim 14, where applying the second complex to the sample detection region is by exposing the second complex to a magnetic field to retain the second complex next to the first electrode.

16. The method of claim 1, wherein the substrate is a portion of the sample detection region of the first electrode.

17. The method of claim 1, wherein the oxidation-reduction reaction comprises a hydrogen evolution reaction (HER).

18. The method of claim 1, wherein the light source comprises a laser diode or a light emitting diode.

19. The method of claim 18, wherein the laser diode or light emitting diode emits a light having a wavelength between 400 nm and 680 nm.

20. A method of signal amplification coupled with detection of a target analyte present in a fluid sample, the method comprising:
providing a plurality of magnetic beads to a first fluid sample, wherein the plurality of magnetic beads comprises first binding moieties that specifically bind to the target analyte;
allowing the first binding moieties of the plurality of magnetic beads to bind to the target analyte in the first fluid sample;
transferring the plurality of magnetic beads from the first fluid sample to a second fluid sample, wherein the second fluid sample comprises a plurality of nanoparticles, wherein the plurality of nanoparticles comprise second binding moieties that bind to the target analyte;
allowing the second binding moieties of the plurality of nanoparticles to bind to the target analyte bound to the first binding moieties of the plurality of magnetic beads;
combining the plurality of magnetic beads and the plurality of nanoparticles with an acid medium to obtain a third fluid sample;
providing the third fluid sample to a sample detection region of a first electrode, wherein the first electrode is electrically coupled to a potentiostat;
irradiating the third fluid sample with a light source to modify reactions;
inducing an oxidation-reduction reaction involving hydrogen ions and the nanoparticles in the third fluid sample; and
monitoring an output of the potentiostat to determine a presence or quantity of the target analyte in the third fluid sample.

21. The method of claim 20, wherein transferring the plurality of magnetic beads comprises:
immersing a sheath within the first fluid sample;
placing a magnet within the sheath such that the plurality of magnetic beads adhere to the sheath;
removing the sheath containing the magnet from the first fluid sample;
immersing the sheath containing the magnet in the second fluid sample; and
removing the magnet from the sheath to release the plurality of magnetic beads to the second fluid sample.

22. The method of claim 20, wherein the third fluid sample is exposed to a magnetic field to retain the plurality of magnetic beads in the third fluid sample next to the first electrode.
